(19) United States
(12) Patent Application Publication
(10) Pub. No.: US 2005/0216087 A1
(43) Pub. Date: Sep. 29, 2005

(54) Disk repair structures for positioning disk repair material

(75) Inventors: James F. Zucherman, San Francisco, CA (US); Ken Y. Hsu, San Francisco, CA (US)

Correspondence Address: FLIESLER MEYER, LLP, FOUR EMBARCADERO CENTER, SUITE 400, SAN FRANCISCO, CA, 94111, US

(73) Assignee: St. Francis Medical Technologies, Inc., Alameda, CA (US)

(21) Appl. No.: 11/002,168

(22) Filed: Dec. 2, 2004

Related U.S. Application Data
(60) Provisional application No. 60/534,316, filed on Jan. 5, 2004.

(51) Int. Cl. A61F 2/44

(52) U.S. Cl. 623/17.16

(57) ABSTRACT

The present invention is directed to a device that can be placed between two adjacent vertebrae, and that is used to repair an injury or defect in the anulus of the intervertebral disk. The implant is characterized by having a flexible structure anchored to the vertebral bone, the flexible structure connected with a patch held in place over the injury or defect. The flexible structure has a hollow interior space which can sustain inside it a hydrogel cushion. The hydrogel cushion acts as a shock absorber for the spine, maintains height of the intervertebral disk space, and prevents further disk herniation due to the narrowing of the intervertebral disk space.

### PRIORITY CLAIM

U.S. Provisional Patent Application No. 60/534,316 entitled DISK REPAIR STRUCTURES FOR POSITIONING DISK REPAIR MATERIAL, James F. Zucherman et al, filed Jan. 5, 2004 (Attorney Docket No. KLYCD-05005US1).

### CROSS-REFERENCE TO RELATED APPLICATIONS

The present application is related to the following United States Patent Application No. XX/XXX,XXX filed on XX/XX/2004, Attorney Docket No. KLYCD-05005US2, filed concurrently with the instant application, and incorporated fully by reference.

### FIELD OF THE INVENTION

This invention relates to a prosthetic vertebral disk repair implant and method.

### BACKGROUND OF THE INVENTION

The spinal column is a biomechanical structure composed primarily of ligaments, muscles, vertebrae and intervertebral disks. The biomechanical functions of the spine include: (1) support of the body, which involves the transfer of the weight and the bending movements of the head, trunk and arms to the pelvis and legs; (2) complex physiological motion between these parts; and (3) protection of the spinal cord and nerve roots.

The intervertebral disk plays an important role in the biomechanical structure of the spine. It cushions the vertebrae and allows for controlled motions of these bones. An intervertebral disk has two components: (1) the nucleus pulposus, or “nucleus”; and (2) the anulus fibrosis, or “anulus.” The disk is positioned between two vertebral endplates located between adjacent vertebrae.

Each endplate creates an intermediate zone between the flexible disk and the rigid bone of the vertebrae. An endplate consists of thin cartilage overlying a thin layer of hard cortical bone. The hard cortical bone of the endplate is connected with cancellous bone of the vertebrae, which is spongy and vascularized.

The anulus is a tough, fibrous ring that has 15-20 overlapping layers that together are resistant to torsion. The ring connects adjacent vertebrae. It also houses the nucleus pulposus.

The nucleus is a gel-like substance that is high in water content. It helps maintain the shape of the anulus without decreasing its flexibility. When a force acts upon adjacent vertebrae, the nucleus moves with the anulus.

Trauma or disease may displace or damage the spinal disk. A disk herniation occurs when the anulus fibers are weakened or torn and the nucleus becomes permanently bulged, distended, or extruded out of its normal space within the confines of the anulus. The herniated or so-called “slipped” nucleus can compress a spinal nerve, causing leg pain, loss of muscle control, or even paralysis. Also, as the disk degenerates, the nucleus loses its water binding ability and deflates, which decreases the height of the nucleus. In turn, because of the decrease in height of the nucleus, the anulus buckles. In regions of buckling, either circumferential or radial anulus tears may occur, potentially resulting in persistent and disabling back pain. Back pain may be compounded by adjacent, ancillary spinal facet joints which are forced into an overriding position from the buckling of the anulus.

Degenerated, diseased, or traumatized disks prevent people from working and can severely impact the lives of patients and their families. The pain associated with such conditions often is treated with medication, surgery, or both. Of course, it is desirable to eliminate the need for major surgery for all individuals, particularly the elderly. Therefore, an easily implantable prosthetic is needed for sealing and promoting healing of injuries or defects in the anulus to prevent recurrence of disk herniation and the resulting impingement of nerves and clinical sequelae.

### BRIEF DESCRIPTION OF THE DRAWINGS

FIG. 1 is a side view of an embodiment of the invention

FIG. 2A is a side cut-away view of an embodiment of the invention.

FIG. 2B is a side cut-away view as in FIG. 2A, except that the embodiment is anchored to the top of two adjacent vertebrae.

FIG. 3 is a side view of yet another embodiment of the invention.

FIG. 4 is a diagram presenting an embodiment of the method of the invention in flow chart form.

FIG. 5A depicts a method of the invention.

FIG. 5B depicts a method of the invention.

FIG. 5C depicts a method of the invention.

### DETAILED DESCRIPTION OF THE PREFERRED EMBODIMENTS OF THE INVENTION

Embodiments of the present invention relate to a prosthetic intervertebral spinal implant for repairing the anulus fibrosis and nucleus pulposis of a damaged or injured intervertebral disk. The implant also serves to cushion impact on the spine. Embodiments of the present invention concern a flexible structure with an internal hollow space that can be anchored to a vertebral bone, and enclose a hydrogel cushion to replace, in whole or in part, the nucleus. The implant sustains in place an anulus patch over an incision, injury, or defect in the anulus of an intervertebral disk. The disclosed embodiments of the invention thus function to replace the nucleus pulposus, which prevents further narrowing of the intervertebral disk space and destabilization of the spine. The embodiments further inhibit recurrent herniation by repairing the weakened anulus and promoting healing of the injured site.

Embodiments of the present invention include a prosthetic intervertebral disk implant for implantation to repair an incision, injury, or defect in the anulus and to prevent narrowing of the intervertebral disk space. The implant is positioned inside the intervertebral disk space, which is defined by the bone endplates of two adjacent vertebrae. In a preferred embodiment, part of the implant is a hydrogel cushion which is encased in a flexible wire structure having a hollow interior space. The hydrogel cushion replaces the nucleus in whole or in part. In addition to its structural role, the hydrogel also can contain therapeutic materials capable of promoting healing of the damaged disk, which are slowly diffusible from the hydrogel through a semi-permeable membrane encasing the gel.

The hydrogel cushion can be inserted in different ways. It can be inserted pre-filled into the hollow internal space of the flexible wire structure. Alternatively, it can be filled after insertion, for ease of implantation. A further alternative is to use a “conditioned” hydrogel that will return to its original shape and size after being subjected to compressive loads on the spine. Yet another alternative is to use a dehydrated gel that can be hydrated after implantation.

In addition to housing the hydrogel cushion, the flexible wire structure further sustains in position a patch over an incision, injury, or damaged site on the anulus. The patch promotes healing of the site. The disclosure further provides a method for implanting the implant.

The implant has a substantially cylindrical flexible wire structure, which has a hollow interior space. The substantially cylindrical flexible wire structure further has a first end and a second end along a longitudinal axis. At the first end, the flexible wire structure is connected with a bone anchor. A hydrogel cushion is encased within the hollow interior space of the flexible wire structure, to serve as a substitute for the damaged nucleus material. At the second end along the longitudinal axis, the flexible wire structure is adapted to receive and engage with an anulus patch after the hydrogel cushion is encased within the flexible wire structure. The second end also is adapted to receive the hydrogel cushion.

It is within the scope of the invention for the flexible wire structure to be made of different wire configurations. By way of example only, it can be made of wires in a weave or mesh. In an alternative embodiment, the flexible wire structure may also be made of a plurality of wires oriented in substantially the same direction, i.e., running from one end of the cylinder to the other, substantially along the longitudinal axis. In an additional alternative embodiment, the substantially cylindrical shape of the flexible wire structure is made of a spiral of at least one wire.

The wire of the flexible wire structure can be made of nitinol, aluminum, stainless steel, nylon, polypropylene suture material, or another flexible, biocompatible material.

In a preferred embodiment, the bone anchor is a bone screw, connected with the first end of the flexible wire structure. However, other bone anchors are also within the scope of this disclosure. The bone anchor is used to anchor the implant in the intervertebral disk space, after the implant is positioned inside that space so that the second end of the flexible wire structure is sustained in place over the injury or defect in the anulus. A screwdriver or other tool can be used to drive a bone screw into the vertebral bone endplate and into the cortical bone. Other varieties of bone anchors will employ other appropriate tools. For the screwdriver or other tool to be able to reach the bone screw or anchor, the anulus patch may be at least partially unattached until after anchoring and placement of the hydrogel cushion, to allow the tool to reach the bone screw or anchor. Alternatively, the anulus patch can be partially folded back.

The hydrogel cushion replaces in whole or in part the herniated nucleus material that is excised prior to implanting the prosthetic disk. As such, the hydrogel cushion prevents narrowing of the intervertebral space, while also cushioning impact on the spine. It should be understood that the hydrogel can be encased in a semi-permeable membrane. The gel inside the membrane can contain therapeutic materials, including but not limited to hormones, neurotransmitter peptides, anti-inflammatory substances, neurotropic factors, and other materials that serve to reduce inflammation, reduce pain, incite repair of anular fibers or nuclear tissue. Such therapeutic materials are slowly diffusable through the semipermeable membrane encasing the hydrogel.

The hydrogel cushion first can be conditioned with a series of compressive loads, so that the hydrogel returns to its desired shape and size following the application and removal of compressive loads once the hydrogel is implanted, and also potentially hydrated. Alternatively, to facilitate insertion, the hydrogel cushion can be filled after implantation. Another alternative is to implant the hydrogel already filled, but not conditioned. It is further within the scope of this disclosure to implant a hydrogel that can be hydrated after implantation. Once skilled in the art will also appreciate that the hydrogel can be encased within a constraining jacket.

The anulus patch can be attached after anchoring the implant and inserting the hydrogel cushion into the hollow interior space of the flexible wire structure. Alternatively, it can be attached partially, before anchoring, with the remainder waiting until after anchoring and insertion of the hydrogel cushion. Further, the anulus patch can be fully attached to the wire structure and be folded out of the way to facilitate the securing of the anchor into the vertebra. The anulus patch can further be shaped like a plug to seal the damaged site of the anulus.

The flexible wire structure can engage the anulus patch with hooks at the second end of the implant. The hooks either can be connected with the wires at the second end, or they can be continuous with them. The hooks further connect the anulus patch and flexible wire structure with the healthy anulus tissue around the damaged site. Alternatively, the patch can associate with the flexible wire structure via loops connected with or extending from the second end of the flexible wire structure which loops are adapated to receive sutures. The sutures penetrate the anulus patch and the anulus tissue.

The anulus patch can remain on the outside of the anulus. Once the implant is positioned and the hydrogel inserted—either filled, filled after insertion, or hydrated after insertion—the anulus patch is secured to the anulus. However, an anulus patch that is positioned on the interior wall of the injured or defective anulus is also within the scope of this disclosure. Further, the disclosure contemplates the use of a patch or plug that promotes tissue growth over and around the patch or plug to scar the injured or defective anulus and permanently repair the injury or defect. For instance, the patch or plug can be made of a wire or plastic mesh, suture material, or other scarring agent, or other appropriate agent that promotes tissue growth.

The preferred method for implanting the prosthetic intervertebral disk implant can use the actual injury site, if one exists, as the point of insertion and positioning of the implant. This approach obviates the need to damage the anulus further with additional incisions for inserting the implant. A cannula with a stylus first is inserted through an incision in the skin. Alternatively, nested cannula are used to expand gradually the point of insertion so that the point of insertion is able to accommodate a cannula of sufficiently large diameter to house the implant and any tools necessary in the disclosed method of implantation.

A device, such as an automated Nucleotome® anterior hand-operated tissue cutter, is inserted through the cannula and used to excise any herniated nucleus material. The device is then withdrawn and the implant is placed inside the cannula, with the bone anchor end positioned to be inserted first, followed by the flexible wire structure. The hydrogel may not yet be encased in the hollow interior space of the flexible wire structure, since a tool is to be inserted into that space to engage the anchoring means with the vertebral bone endplate in the intervertebral space. A plunger is used to urge the implant through the cannula and through the insertion site. Alternatively, any other tool, including the tool that will be used to anchor the implant can be used to urge the implant through the cannula.

A tool, such as a screwdriver, is used to drive the bone anchor into a vertebral bone endplate. This disclosure contemplates using either the upper or lower bone endplate of two adjacent vertebrae on either side of an injured or defective disk.

Once the screwdriver or other tool is withdrawn, the hydrogel cushion is introduced into the cannula and urged through the damaged site of the anulus and into the hollow interior space of the flexible wire structure which resides in the intervertebral space. It is within the scope of the invention for the hydrogel cushion already to be filled at the time it is positioned with the flexible wire structure, or to be partially filled. It is also within the scope of this invention to fill the hydrogel after implantation, or to hydrate a dehydrated hydrogel after implantation. Also, as described above, the hydrogel cushion can be “conditioned” to return to a particular shape after compressive loading. The hydrogel cushion further can be encased inside a constraining jacket and urged through the cannula.

After the hydrogel cushion is positioned, the anulus patch is introduced into the cannula with a tool. Hooks that are either connected with or extended from the second end of the flexible wire structure (i.e., distal to the end of the flexible wire structure that is connected with the bone anchor) are adapted to receive the anulus patch and engage it, so that the anulus patch covers the damaged site of the anulus. The hooks further engage the healthy tissue around the damages site to sustain the anulus patch and the flexible wire structure in position. Alternatively, the anulus patch can be sutured onto the tissue around the injury or defect through loops extending from or continuous with the second end of the flexible wire structure.

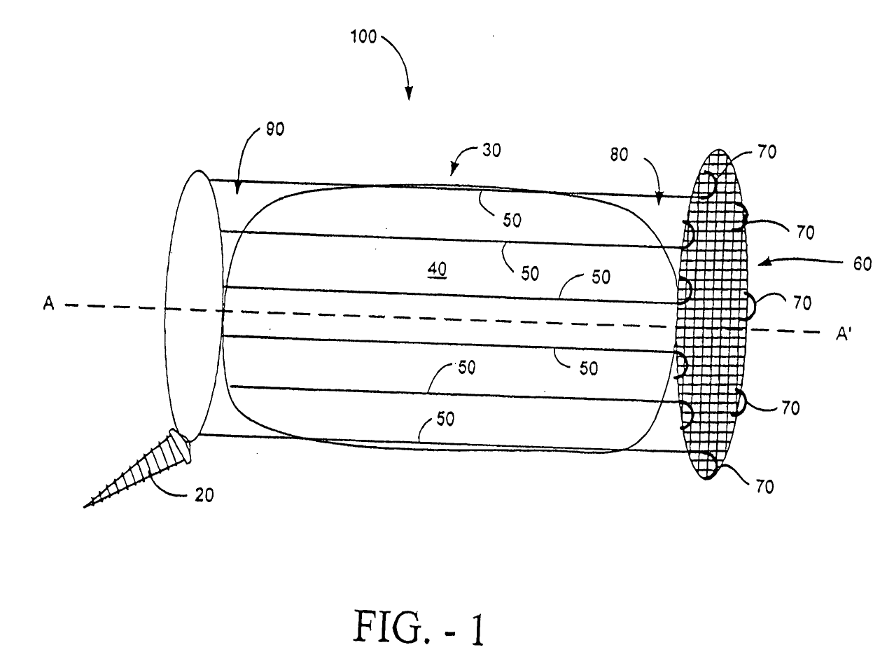

### Embodiment of FIG. 1

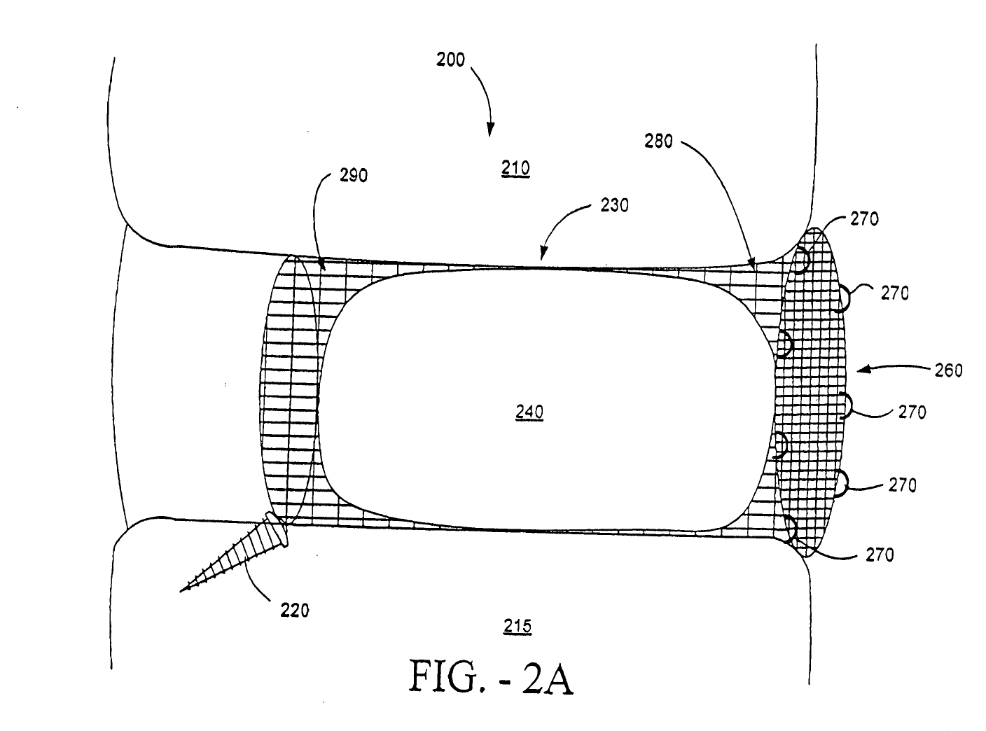

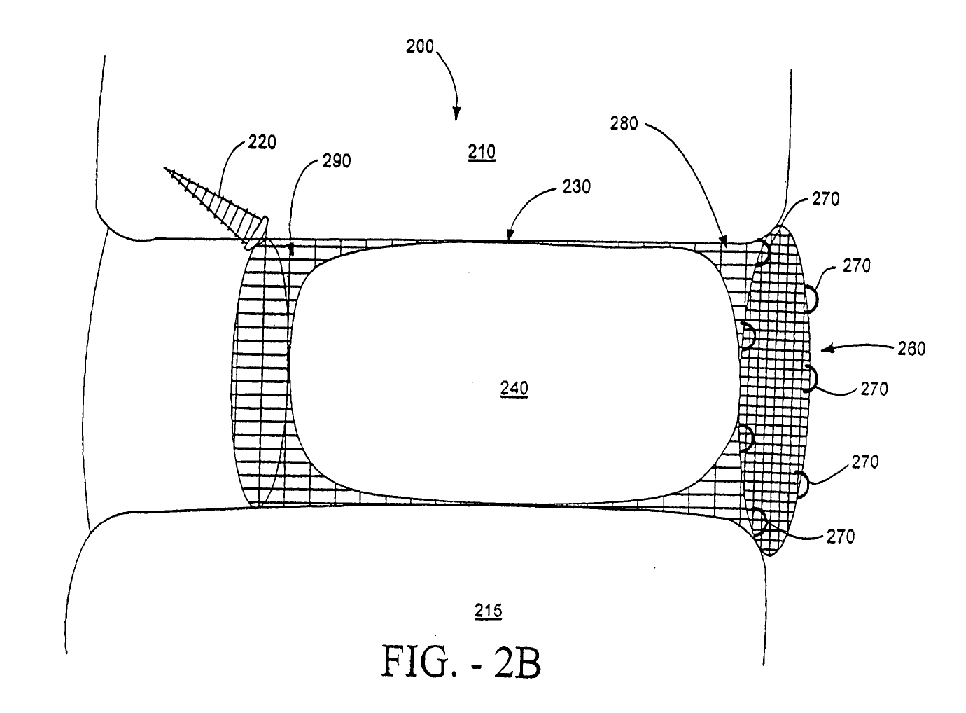

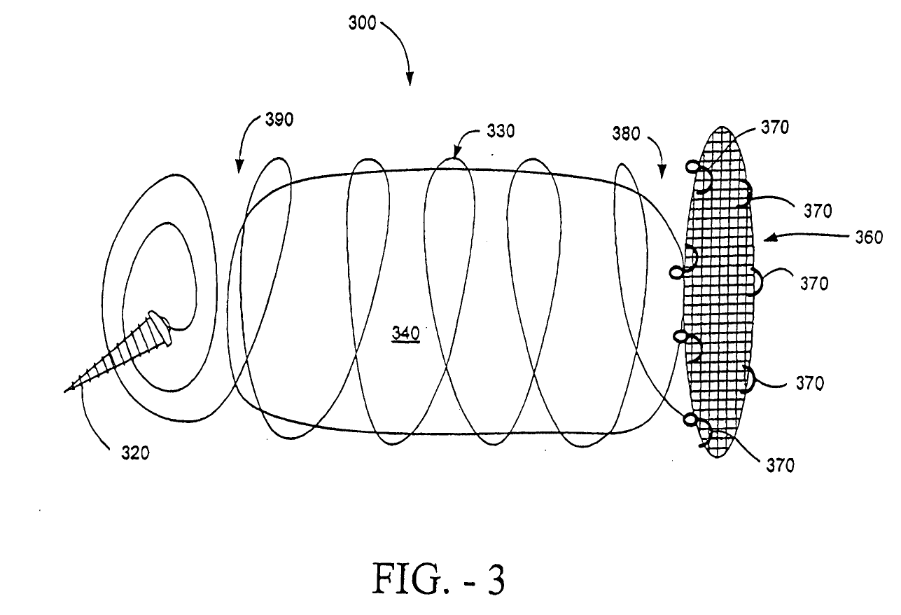

One preferred embodiment 100 of a prosthetic intervertebral spinal implant for repairing an intervertebral disk is shown in FIG. 1. In this embodiment, the implant 100 comprises a flexible wire structure 30 that is substantially cylindrical and has an interior hollow space 90. The flexible wire structure 30 further has a longitudinal axis, labeled here as A-A′. The plurality of wires 50 comprising the flexible wire structure 30 can have various configurations. For example, in FIG. 1, the plurality of wires 50 runs substantially parallel to the longitudinal axis A-A′. Other configurations also within the scope of this disclosure are depicted in FIGS. 2A, 2B, AND 3. The plurality of wires 50 can be made of a flexible biocompatible metal such as nitinol, aluminum, titanium, or stainless steel.

The prosthetic intervertebral spinal implant 100 further comprises an anchoring means 20, connected with the flexible wire structure 30 at a first end of the longitudinal axis A-A′, to anchor the implant 100 to a vertebral bone endplate in the intervertebral disk space. The implant 100 is anchored to the endplate once the flexible wire structure 30 is positioned within the intervertebral space such that the second end 80 of the flexible wire structure 30 is adjacent to the damage site on the anulus.

The anchoring means 20 depicted in FIG. 1 is a bone screw, but the disclosure further encompasses bone anchors and other types of anchoring means including, by way of example only, bone pins and bone sutures that can penetrate the vertebral bone endplate and into the cortical bone. The anchoring means 20 can be made of a biocompatible metal, including nitinol, titanium, and stainless steel. The anchoring means 20 may also be made of a resorbable material.

The implant 100 further comprises a hyrdogel cushion 40. As mentioned, the flexible wire structure 30 has a hollow interior space 90. In one embodiment, the hollow interior space 90 can accept the hydrogel cushion 40 once the bone anchor 20 is engaged. A tool is required to reach the anchoring means 20 through the hollow interior space 90 of the flexible wire structure 30, and to drive the anchoring means 20 into the vertebral bone endplate. If the hydrogel cushion 40 already were in place, it could take longer to secure the bone screw.

The hydrogel cushion 40 serves various functions. It anchors the implant 100 within the intervertebral disk space. Moreover, the hydrogel cushion 40 replaces either in whole or in part the herniated nucleus material that can be excised to accommodate the implant 100. As such, the disk prosthesis assumes the shock absorbing function of the damaged nucleus and cushions shocks and compressive loads on the spine. The hydrogel cushion 40 also preserves the height of the intervertebral disk space and prevents further narrowing and loss of mobility. In this regard, the prosthetic implant 100 also prevents further weakening of the anulus.

The hydrogel cushion 40 can be conditioned with a series of compressive loads to assume its original shape and size following the application and removal of compressive loads on the spine, once the hydrogel is implanted. Conditioning and storage of the hydrogel cushion 40 prior to implantation has been fully described and the details need not be repeated here.

The constraining jacket, which must be flexible without stretching, is made of a high molecular weight, high tenacity polymeric material with pores. Several examples of such materials have been described in the art, and include high molecular weight polyethylene, polyester, or any other high molecular weight, high tenacity polymeric material, as well as carbon fiber yards, ceramic fibers, metallic fibers, etc. The constraining jacket maintains the desired shape of the hydrogel cushion 40 by preventing horizontal expansion and consequently, avoiding additional stress on the anulus, as described. The pores in the constraining jacket allow bodily fluids to interact with the hydrogel cushion 40 so that it can imbibe fluids and expand so that its characteristics more closely imitate those of a natural intervertebral disk nucleus.

Alternatively, the hydrogel cushion 40 can also be filled after it is implanted. The hydrogel further can incorporate therapeutic agents. The hydrogel cushion 40 in this alternative embodiment has a semi-permeable membrane that is chemically and biologically inert, and filled with fluid containing therapeutic materials. The therapeutic materials are selected from the group consisting of hormones, neurotransmitter peptides, anti-inflammatory substances, neurotropic factors, pain medications, and healing stimulants. The fluid inside hydrogel cushion 40 can be an aqueous thixotropic gel having a viscosity and velocity-shear behavior approximating the natural characteristics of the nucleus. The hydrophilicity of the gel allows the hydrogel cushion 40 to take up and expel water and assume the function of a normal intervertebral disk.

It is further to be appreciated that the hydrogel cushion 40 can be pre-filled prior to implantation, partially filled prior to implantation, or filled after implantation. The hydrogel cushion 40 can also contain hydrogel in dehydrated form upon implantation, which is later hydrated after implantation.

The implant 100 further comprises an anulus patch 60. The purpose of the anulus patch 60 is to seal the damaged site of the anulus to prevent further herniation at the weakened site. The anulus patch 60 further serves to promote healing of the damaged site to prevent recurrence of the disk herniation. It also prevents ejection of the hydrogel cushion 40 from the intervertebral disk space. The anulus patch 60 can be made from materials selected from the group consisting of wire mesh, plastic mesh, scarring agents, or hydrogel. In an alternative embodiment, an anulus plug can be used in place of an anulus patch.

The implant 100 further comprises a plurality of connecting means 70 to receive the anulus patch 60 and engage it with a second end 80 of the flexible wire structure 30 at a second end of the longitudinal axis A-A′. Once the hydrogel cushion 40 is positioned inside the hollow interior space 90 of the flexible wire structure 30, the anulus patch 60 can be connected with the second end of the flexible wire structure 80 via the connecting means 70. As depicted in FIG. 1, the connecting means 70 can be a plurality of hooks extending from the plurality of wires 50 at the second end 100 of the flexible wire structure 30. The hooks 70 pierce the anulus patch 60 and engage it with the open second end 80 of the flexible wire structure 30 to seal the damaged site of the anulus and promote healing of the damaged tissue. The hooks 70 either can be continuous with the plurality of wires 50 at the second end 80, or be separate hooks that attach to the plurality of wires 50.

Alternatively, the connecting means 70 can be a plurality of loops extending from the plurality of wires 50 at the second end 80 of the flexible wire structure 30. The loops are adapted to receive sutures which penetrate the anulus patch 60 and the tissue of the anulus around the damaged site, to sustain the anulus patch 60 over the damaged tissue. A combination of hooks and loops also is contemplated to be within the scope of this disclosure.

### Embodiments of FIGS. 2A and 2B

FIGS. 2A AND 2B depict side cut-away views of two different implantation results of a further embodiment 200 of the disclosed implant.

In this embodiment, the flexible wire structure 230 comprises a wire weave which, if a tight weave, can be a wire mesh. As above, the wire can be made of material selected from the group consisting of nitinol, aluminum, stainless steel, and titanium. FIG. 2A demonstrates that the implant 200 can be anchored into the lower 220 of two adjacent vertebrae, and FIG. 2B illustrates that the implant 200 can be anchored alternatively to the top 210 of two adjacent vertebrae, within the intervertebral disk space.

As above, the hydrogel cushion 240 is enclosed within the flexible wire structure 230, which is positioned within the intervertebral disk space. The second end 280 of the flexible wire structure 230 abuts the damaged site in the anulus and is adapted to receive the anulus patch 260 with a connecting means 270. Also as above, a plurality of hooks engage the anulus patch 260 to seal the damaged site in the anulus and promote healing of the anulus tissue to prevent recurrent herniation. However, it is also contemplated that loops extending from the wires of the second end 280 of the flexible wire structure 230 can be used to engage the anulus patch 260. The loops are adapted to accept sutures that penetrate the anulus patch 260 and the tissue of the anulus around the damaged site to seal the damaged tissue and promote healing.

### Embodiment of FIG. 3

A further embodiment 300 of the disclosure is depicted in FIG. 3. In this embodiment 300, the flexible wire structure 330 comprises at least one wire that spirals to form a substantially cylindrical shape and connects with the bone anchor 320 at the first end. As in the other embodiments described, the anulus patch connecting means 370 can be hooks extending from the second end 380 of the flexible wire structure 330. The hooks can be attached to the second end 380 of the flexible wire structure 330 as depicted in this embodiment 300. Alternatively, they can be loops extending from the second end 380, adapted to receive sutures that sustain the anulus patch 360 over the damaged site in the anulus and engage the anulus patch 360 with the anulus around the damaged anulus tissue. Also as with the other embodiments, the hollow interior space 390 of the flexible wire structure 330 is adapted to contain the hydrogel cushion 40. The hydrogel cushion 340 can be filled prior to or after insertion into the hollow interior space 390. It can contain therapeutic materials as described above. The hydrogel cushion 340 also can be encased in a constraining jacket, and the constraining jacket can be used as a foundation to fasten or sew the hydrogel to the spiral cage. It can further be dehydrated upon implantation for hydration after implantation. In a preferred embodiment, the hydrogel cushion 40 is conditioned with a series of compressive loads prior to positioning inside the flexible wire structure 330.

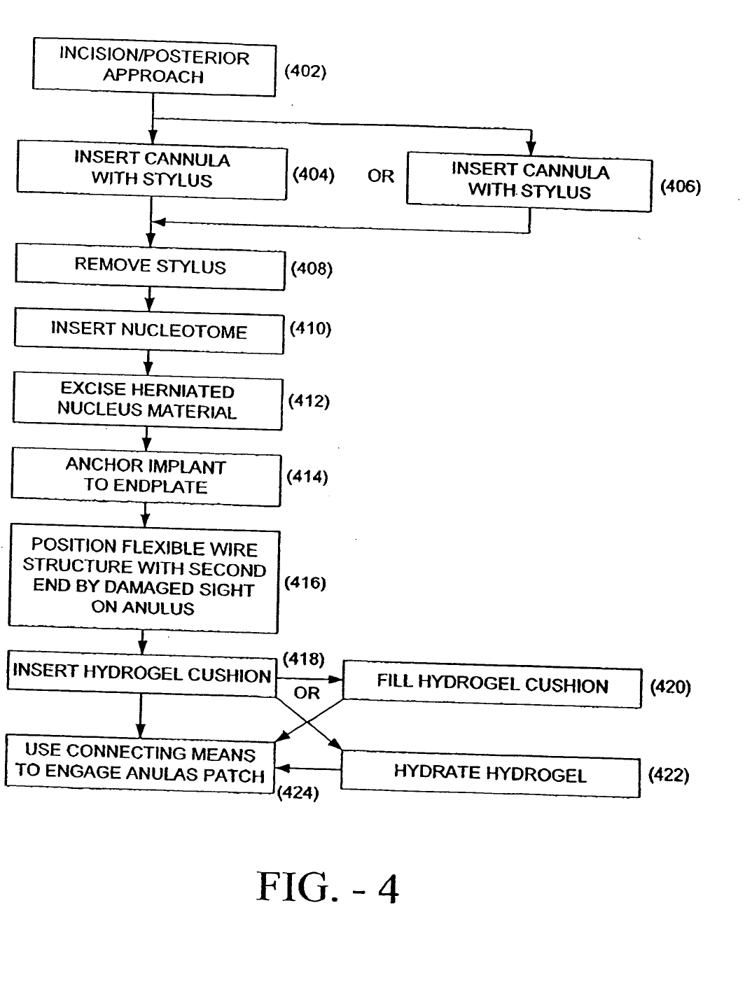

### Embodiment of FIG. 4

An embodiment of a method for implanting a spinal disk repair implant 400 is depicted in flow chart format in FIG. 4. First, an incision or puncture is made using, for example, a posterior approach 402. A cannula is inserted with a stylus 404 and the cannula moved into position at the site of the injury or defect to the anulus. The stylus is then removed 408, and a Nucleotome® is inserted into the cannula 410. The Nucleotome® tool includes a guillotine blade that can be used to excise herniated nucleus material 412.

As an alternative to a cannula/stylus 404, nested cannulae and a guide wire can be used to position the cannula and widen gradually the incision and to access the intervertebral disk space 406. The guide wire is inserted first, followed by successively wider-bore cannulae. The smaller interior cannulae are then removed, as well as the guidewire, and a larger operating space is available through the broadest cannula. The Nucleotome® is then inserted 410 and applied to remove herniated disk material, as above 412.

Once the herniated nucleus material is removed 412, the Nucleotome® is extracted from the cannula and the implant can be anchored 414. An implant essentially as described above is inserted into the cannula with the bone anchor inserted first, so that the bone anchor is the first part to penetrate the anulus and enter the intervertebral disk space. The anulus patch can be partially connected with the connecting means at the second end of the flexible wire structure, if partial attachment permits use of a tool to urge the implant along the cannula and to engage the anchoring means with the vertebral bone endplate, and further also allows later insertion of the hydrogel cushion into the hollow interior space of the flexible wire structure.

The implant is caused to enter the intervertebral disk space via the damaged site of the anulus that is to be repaired. Inserting the implant through the same part of the anulus that already has been damaged is beneficial to the patient, since it avoids further injury to the anulus, which would result from making additional incisions in the fibrous tissue layers. For example, cutting flaps out of the anulus could cause a loss of integrity of its fibrous layers. Further injury may result from any weakening in the anulus, and the possibility of its healing completely would be reduced.

A tool is inserted into the cannula to cause the anchoring means to engage with a vertebral bone endplate 414. If the anchoring means is a bone screw, then the tool is a screw driver with a head adapted to engage the bone screw and drive it into the bone endplate. Alternatively, the anchoring means can be a type of bone anchor or bone suture that is able to penetrate the bone endplate.

After the anchoring means is engaged, the tool used to drive the anchoring means into the bone endplate is removed from the cannula. The flexible wire structure of the implant is positioned to receive the hydrogel cushion through the cannula 416. As set forth above, the hydrogel cushion can be filled prior to insertion into the flexible wire structure, and a filled hydrogel cushion can be conditioned by methods already described which do not bear further elaboration here 418. The hydrogel cushion can also contain therapeutic agents. It is also possible to fill the hydrogel cushion after it is already positioned within the intervertebral disk space, and in particular, inside the flexible wire structure 420. Alternatively, the hydrogel can be implanted as dehydrated and re-hydrated after implantation 422.

Once the hydrogel cushion has been inserted into the hollow interior space of the flexible wire structure 418, and filled or hydrated, if necessary 420, 422, the anulus patch can be connected with the implant at the second end of the flexible wire structure to seal the site of the damage to the anulus so that it can also promote healing at that site 424. As described for the various embodiments, the connecting means can be hooks extending from the wires at the second end of the flexible wire structure 424. Alternatively, the connecting means can be loops that are adapted to receive sutures to connect the anulus patch with the healthy tissue around the defect or injury in the anulus and with the flexible wire structure 424.

Other embodiments already will have the anulus patch partially attached to the flexible wire structure, to allow access by a tool used to engage the bone screw with the bone endplate. In these embodiments, once said tool is removed from the cannula, the anulus patch can be completely connected with the flexible wire structure using hooks connected with or continuous with the second end of that structure, or loops and sutures, as described in detail above.

A tool can be used to position the anulus patch and pierce it with the hooks that are to hold it in position and engage the anulus tissue around the damaged site. Alternatively, the tool can be used to manipulate loops into position to receive sutures that will hold the patch to the anulus and the flexible wire structure.

The cannula is then removed from the incision and the incision is surgically closed.

It is also to be understood that the entire implant including the hydrogel cushion can be inserted all together. In this embodiment the hydrogel cushion can be completely filled, or partial filled or partially hydrated. After the implant is secured to the bone, if the hydrogel cushion is not prefilled, the hydrogel cushion can then be filled or hydrated. With this embodiment, preferably the hydrogel cushion, as well as the anulus patch, can be urged out or the way as a tool is used to secure the implant to, for example, a vertebral body.

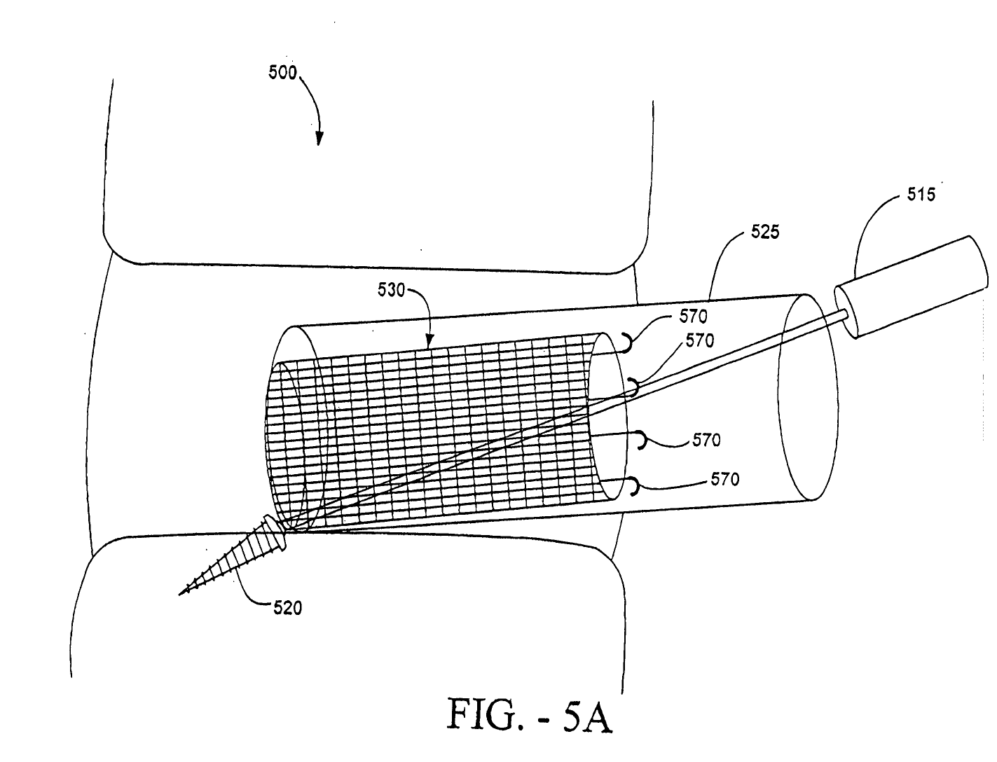

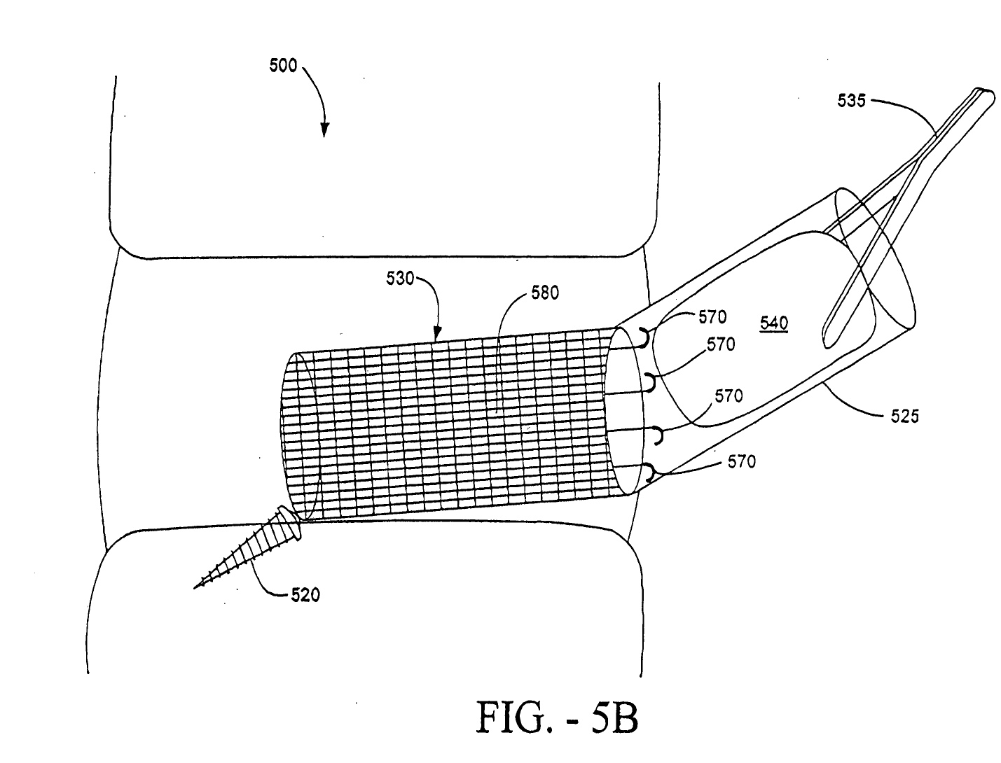

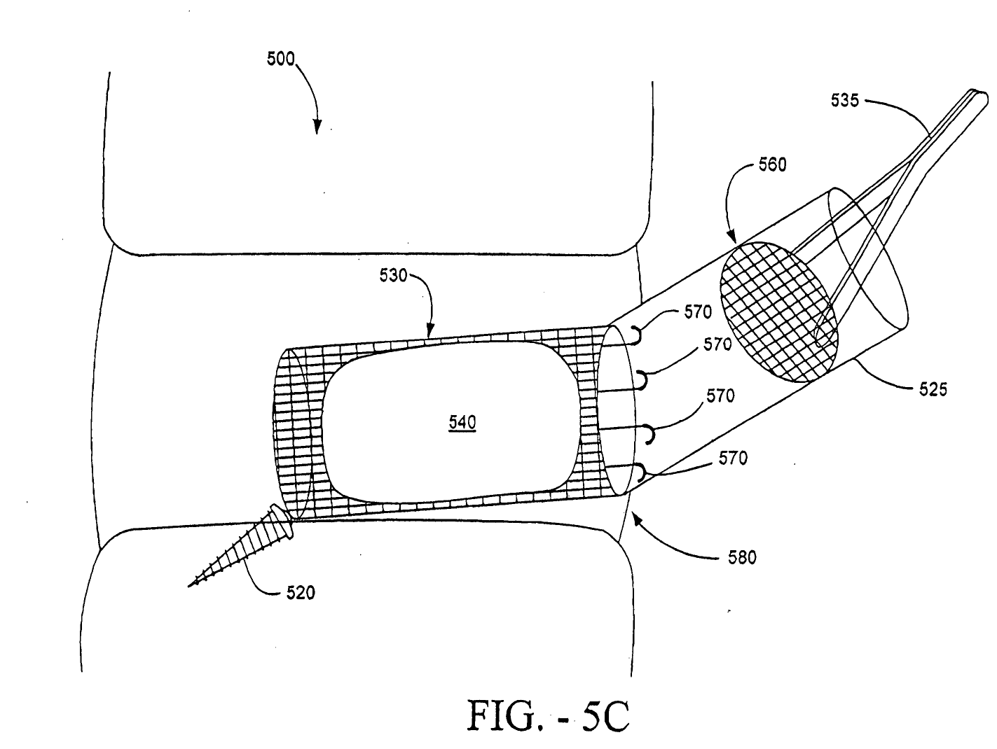

### Embodiment of FIG. 5

The embodiment 500 of FIGS. 5A, 5B, and 5c depict the method described above. In FIG. 5A, an implant as described above is urged down a cannula 525 that has been inserted through an incision or puncture. A screwdriver or other appropriate tool 515 is used to engage the bone anchor with the vertebral bone in the intervertebral disk space. FIG. 5B depicts placement of the hydrogel cushion 540 in the hollow interior space 590 of the wire structure 530 of the implant via the cannula 520, using an appropriate tool 530. Such tool 530 can be a type of forceps that will not puncture or otherwise damage the hydrogel cushion 540. The hydrogel cushion 540 can be sutured in place, if desired.

FIG. 5c illustrates engagement of the anulus patch 560 with the connecting means 570 after the hydrogel cushion 540 is positioned inside the hollow interior space 590 of the wire structure 530, as described in FIG. 5B. An appropriate tool 535, which can be a type of forceps, is used to manipulate the anulus patch 560 and the connecting means 570 to engage the connecting means 570 and the anulus patch 560. The anulus patch 560 will be positioned over the injured, damaged, or otherwise defective site in the anulus. After the anulus patch 560 is secured to the wire structure 530 by the connecting means 570, here depicted as a plurality of hooks at the second end 580 of the wire structure 530, the cannula 525 is withdrawn.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated. It is intended that the scope of the invention be defined by the following claims and its equivalence.

1. An implant for repairing damage to an anulus of an intervertebral disk, the implant comprising:
a first anchor part adapted to anchor the implant to a first vertebra;
a second structure part with a first end and a second end, attached to the first anchor part;
a hollow internal space defined by the second structure part, the hollow internal space can receive a third cushion part;
the third cushion part; and
a fourth patch part that connects by a connector with the second end of the second structure part, the fourth patch part adapted to patch the damage to the anulus.

2. The implant of claim 1 wherein the second structure part comprises a plurality of wires.

3. The implant of claim 1 wherein the connector is a plurality of hooks.

4. The implant of claim 1 wherein the connector is a plurality of loops adapted to receive sutures to secure the fourth patch part to the anulus.

5. The implant of claim 1 wherein the connector includes a plurality of hooks, that can engage the fourth patch part and the anulus, and a plurality of loops, adapted to receive sutures to secure the fourth patch part to the anulus.

6. The implant of claim 2 wherein the plurality of wires is configured as a mesh.

7. The implant of claim 2 wherein the plurality wires is a wire weave.

8. The implant of claim 1 wherein the second structure part is configured as a spiral.

9. The implant of claim 1 wherein the second structure part is configured as a spiral.

10. The implant of claim 1 wherein the first anchor part is a bone screw.

11. The implant of claim 10 wherein the bone screw is made of a material selected from the group consisting of nitinol, titanium, stainless steel, and a resorbable material.

12. The implant of claim 1 wherein the first anchor part is a bone suture.

13. The implant of claim 1 wherein the fourth patch part is an anulus patch made of a material selected from the group comprising plastic mesh, wire mesh, scarring agents, and hydrogel.

14. The implant of claim 1 wherein the fourth patch part is an anulus patch shaped like a plug.

15. The implant of claim 1 wherein the third cushion part is filled with a thixotropic gel.

16. The implant of claim 1 wherein the third cushion part contains therapeutic materials selected from the group consisting of hormones, neurotransmitter peptides, anti-inflammatory substances, neurotropic factors, pain medications, and healing stimulants.

17. The implant of claim 1 wherein the second structure part is made of material selected from the group consisting of nitinol, aluminum, stainless steel, and titanium.

18. The implant of claim 1 wherein the third cushion part is a hydrogel.

19. An implant for repairing damage to an anulus of an intervertebral disk, the implant comprising:
a bone screw that anchors the implant to a vertebral bone endplate;
a structure including a first end and a second end along a longitudinal axis, and connected to the bone screw at the first end;
a hollow internal space defined by the structure;
a hydrogel cushion received through the second end of the wire structure and retained in the hollow internal space; and
an anulus patch secured to the second end of the wire structure and the anulus by at least one connector extending from the second end of the flexible wire structure.

20. The implant of claim 19 wherein the structure comprises a plurality of wires.

21. The implant of claim 19 wherein the connector is a plurality of hooks.

22. The implant of claim 21 wherein the connector is a plurality of loops adapted to accept sutures.

23. A method for implanting a device encasing a cushion to repair damage to an anulus of an intervertebral disk, the method comprising:
inserting a first end of a cannula in an incision;
positioning the first end of the cannula by a defect in the anulus;
excising herniated disk tissue;
urging the device through the cannula and into the intervertebral disk space;
anchoring the device with an anchor;
positioning the device and a cushion in the intervertebral disk space;
patching the damaged anulus tissue; and
closing the incision.

24. The method of claim 23 wherein the inserting step further comprises using a stylus within the cannula.

25. The method of claim 23 wherein the anchoring step includes engaging a bone anchor at the first end of the device with a vertebral bone endplate.

26. The method of claim 23 wherein the step of inserting a cannula further includes inserting a plurality of nested cannulae along a guide wire for monitoring the surgery by flouroscopy.

27. The method of claim 23 including the step of inserting the cushion filled with a hydrogel.

28. The method of claim 23 wherein the method further includes the step of filling the cushion with a hydrogel after implanting the device.

29. The method of claim 23 wherein the method further comprises filling the cushion with a hydrogel, the hydrogel containing at least one therapeutic agent selected from the group consisting of hormones, neurotransmitter peptides, anti-inflammatory substances, neurotropic factors, pain medications, and healing stimulants.
